(19) United States
(12) Patent Application Publication
(10) Pub. No.: US 2026/0224114 A1
(43) Pub. Date: Aug. 6, 2026

(54) METHODS AND SYSTEMS FOR INFECTION DETECTION

(71) Applicant: KONINKLIJKE PHILIPS N.V., EINDHOVEN (NL)

(72) Inventors: Emmanuele Salvati, Cambridge, MA (US); Bryan Conroy, Cambridge, MA (US); Sara Mariani, Cambridge, MA (US); lkaro Garcia Araujo da Silva, Cambridge, MA (US)

(21) Appl. No.: 19/152,115

(22) PCT Filed: Jan. 30, 2024
(86) PCT No.: PCT/EP2024/052118

§ 371 Date: Jul. 30, 2025

Related U.S. Application Data
(60) Provisional application No. 63/444,586, filed on Feb. 10, 2023.

(51) Int. Cl. A61B 5/01; A61B 5/00

(52) U.S. Cl. CPC A61B 5/01; A61B 5/7271; A61B 5/746

(57) ABSTRACT

A method for determining whether the user has an infection including: receiving, from a wearable device, a time series of data representing a physiological variable measured by the wearable device; determining a first parametric function characterizing a distribution of a first portion of the time series of data, at least one of the first set of parameters characterizing a skew of the first parametric function; determining a second parametric function characterizing a distribution of a second portion of the time series of data, at least one of the second set of parameters characterizing a skew of the second parametric function, wherein the first portion of the time series of data represents a baseline window and the second portion represents an observation window; and determining from at least one measure of difference between the first set of parameters and the second set of parameters, whether the user is currently carrying an infection.

### FIELD OF THE DISCLOSURE

The present disclosure is directed generally to methods and systems for early detection of infection.

### BACKGROUND

Early detection of infectious diseases, such as COVID-19, RSV, and influenza, is critical to containing their spread. Often, carriers of these diseases are contagious well before being symptomatic, meaning that the disease is spread before the carrier is aware of the infection and can self-isolate. Early detection of infections is difficult, however, because symptoms are often transient and their duration varies across individuals, making it difficult to model in a typical machine learning classification algorithm. The magnitude of symptoms can also be marginal in certain cases, which might lead to marginal classification accuracy. Further, determining a healthy—i.e., not infected—state of an individual can have many confounding factors (such as the user failing to wear a particular wearable consistently or the user having back-to-back infections), especially if data is collected over a short period of time.

### SUMMARY OF THE DISCLOSURE

Accordingly, there is a continued need for early detection of infectious disease, which can account for varying, transient, and marginal symptoms during the early stages of infection. Various embodiments and implementations described in this disclosure are directed toward a method or system for early detection of infectious disease using data collected regarding one or more physiological variables of the user over time, for example via a wearable device. The system receives the physiological variable data and organizes it into distributions collected over different windows of time, a baseline window and an observation window. The shape of the distribution of the two windows, and particularly the nature of the “tails” of the distributions, are compared to determine whether the user is currently carrying an infection. The comparison of the distribution can be made by characterizing the maximum likelihood fit of each distribution with a parametric function, such as a Skew Normal function. Differences between the parameters of the parametric functions can then be input to a predictive model to detect the early presence of an infection.

According to an aspect, a method for determining, from data collected by a wearable device worn by a user, whether the user has an infection, includes the steps of: receiving, from a wearable device, a time series of data representing a physiological variable measured by the wearable; determining a first parametric function, defined by a first set of parameters, characterizing a distribution of a first portion of the time series of data, at least one of the first set of parameters characterizing a skew of the first parametric function; determining a second parametric function, defined by a second set of parameters, characterizing a distribution of a second portion of the time series of data, at least one of the second set of parameters characterizing a skew of the second parametric function, wherein the first portion of the time series of data represents a baseline window and the second portion represents an observation window; and determining from at least one measure of difference between the first set of parameters and the second set of parameters, whether the user is currently carrying an infection.

In an example, determining a first parametric function, includes: dividing the first portion of the time series of data into a plurality of portions; determining a plurality of parametric functions respectively characterizing a distribution of each of the plurality of the portions, wherein each of the plurality of parametric functions is defined by a set of parameters that characterize, at least, a skew of the parametric function, wherein the first parametric function is determined as a mean or a median of the set of parameters of the plurality of parametric functions.

In an example, the first distribution function and the second distribution function are each skew normal functions.

In an example, the at least one measure of difference between the first set of parameters and the second set of parameters, comprises determining at least one of a Kullback-Leibler divergence, effect size, and earth mover's distance.

In an example, determining whether the user is currently bearing an infection comprises using the at least one measure of difference between the first set of parameters and the second set of parameters as an input to a predictive model.

In an example, the predictive model is trained using data measured from a plurality of users.

In an example, the predictive model is a classification model.

In an example, the first portion of the time series of data is collected over at least three weeks.

In an example, the second portion of the time series of data is collected over at most three days.

According to another aspect, a non-transitory storage medium storing program code that, when executed by a processor, determines, from data collected by a wearable device worn by a user, whether the user has an infection, the program code includes the steps of: receiving, from a wearable device, a time series of data representing a physiological variable measured by the wearable; determining a first parametric function, defined by a first set of parameters, characterizing a distribution of a first portion of the time series of data, at least one of the first set of parameters characterizing a skew of the first parametric function; determining a second parametric function, defined by a second set of parameters, characterizing a distribution of a second portion of the time series of data, at least one of the second set of parameters characterizing a skew of the second parametric function, wherein the first portion of the time series of data represents a baseline window and the second portion represents an observation window; and determining from at least one measure of difference between the first set of parameters and the second set of parameters, whether the user is currently carrying an infection.

In an example, determining a first parametric function, comprises: dividing the first portion of the time series of data into a plurality of portions; determining a plurality of parametric functions respectively characterizing a distribution of each of the plurality of the portions, wherein each of the plurality of parametric functions is defined by a set of parameters that characterize, at least, a skew of the parametric function, wherein the first parametric function is determined as a mean or a median of the set of parameters of the plurality of parametric functions.

In an example, the first distribution function and the second distribution function are each skew normal functions.

In an example, the at least one measure of difference between the first set of parameters and the second set of parameters, comprises determining at least one of a Kullback-Leibler divergence, effect size, and earth mover's distance.

In an example, determining whether the user is currently bearing an infection comprises using the at least one measure of difference between the first set of parameters and the second set of parameters as an input to a predictive model.

In an example, the predictive model is trained using data measured from a plurality of users.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 depicts a system for early detection of an infection, according to an example.

FIG. 2 depicts a flowchart of a method for early detection of an infection, according to an example.

FIG. 3A depicts an example histogram, and the maximum likelihood fits, of the baseline window and observation window temperatures measured for a COVID-19 positive user.

FIG. 3B depicts an example histogram, and the maximum likelihood fits, of the baseline window and observation window temperatures measured for a COVID-19 negative user.

FIG. 4A depicts an example Kullback-Leibler divergence between baseline and observation window fit functions obtained from COVID-19 positive and negative cases.

FIG. 4B depicts an example effect size between baseline and observation window fit functions obtained from COVID-19 positive and negative cases.

FIG. 4C depicts an example earth mover's distance between baseline and observation window fit functions obtained from COVID-19 positive and negative cases.

FIG. 5 depicts a portion of a flowchart of a method for early detection of an infection, according to an example.

FIG. 6 depicts an example set of maximum likelihood fits, respectively fit to temperature values measured over many three-day periods computed between one week and six months prior to a COVID-19 test for a single participant, as well as the mean and median values of the maximum likelihood fits.

### DETAILED DESCRIPTION OF EMBODIMENTS

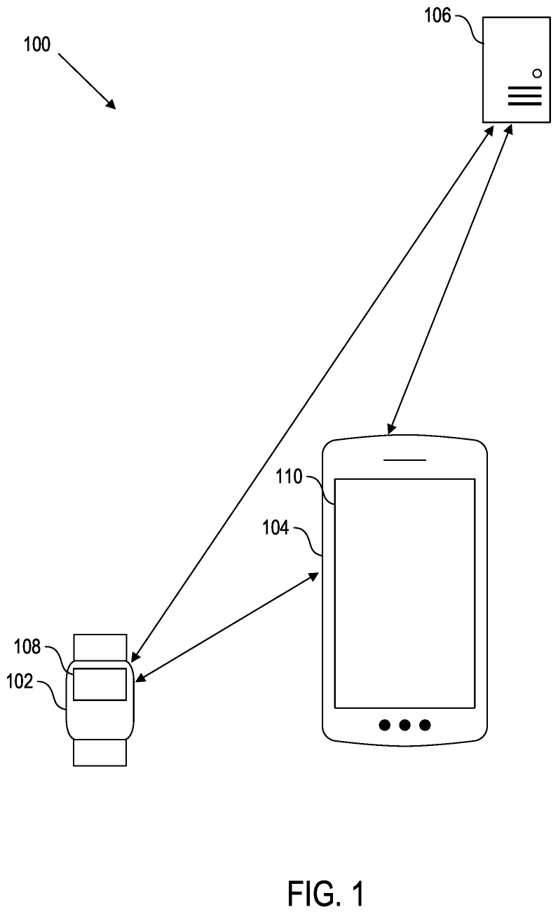

With reference to FIG. 1, a system 100 for early detection of infectious diseases is illustrated according to aspects of the present disclosure. There is shown in FIG. 1 an example system 100. As shown, system 100 includes a wearable device 102, a mobile device 104, and a remote server 106. In various embodiments and implementations, wearable device 102, worn by a user, can detect various physiological variables (such as body temperature, heart rate, oxygen saturation, respiratory rate etc.) and transmit data representing the physiological variables to mobile device 104, which can relay this data to remote server 106. In an alternative example, wearable 102 can transmit the data directly to remote server 106. Remote server 106 can analyze the data to determine whether the user wearing wearable 102 is carrying an infection.

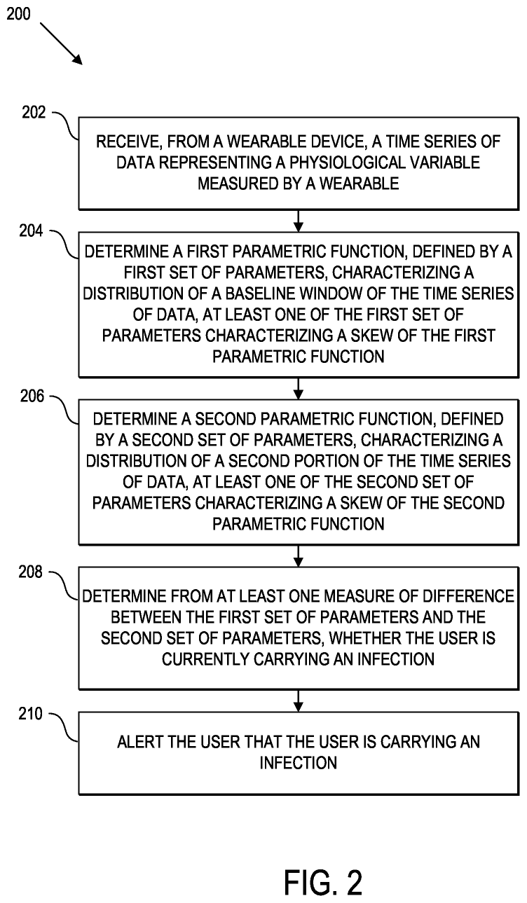

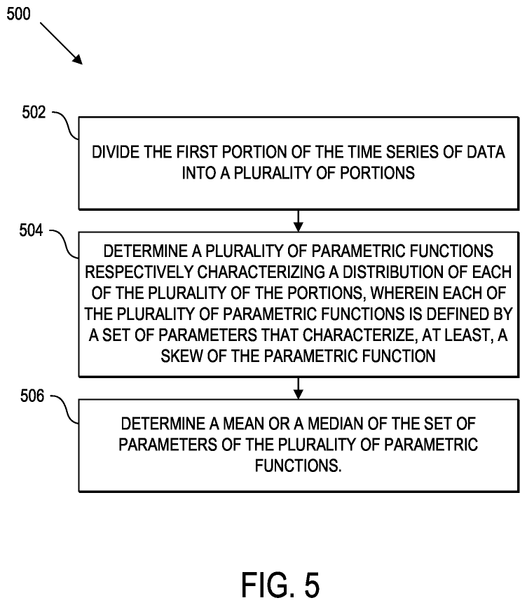

This analysis can comprise: (1) calculating a distribution function of a baseline window of data and a distribution function of an observation window of data; (2) finding a parametric function that defines a maximum likelihood fit of both distribution functions, the parametric function being denoted by parameters that include, at least, a skew of the fit, and (3) finding at least one measure of difference between the parameters of the fit of both distribution functions. Whether a user is carrying an infection is then determined from the measure (or measures) of difference between the parameters of the parametric functions. This step can include inputting the measure(s) of difference into a predictive model, such as a classification model. Applicant has recognized and appreciated that differences in the shape of the distribution functions, and, in particular, the difference in the tails of the distribution functions, are useful for early detection of infection. This method, which will be described in more detail in connection with FIGS. 2 and 5, can account for the marginal and transient symptoms present in the early stages of infection with a high degree of accuracy. Once an infection has been detected, the user can be notified, such as through a display 108 on wearable device 102, or a message (e.g., a notification through a dedicated application) that can be displayed on display 110 of mobile device 104.

Wearable device 102 can comprise any device worn by the user or attached to the user's body suitable for measuring one or more physiological variables useful for accomplishing the early detection of infection, as described in this disclosure. Examples of such wearables include mobile devices that can be worn on the wrist to track physiological variables, such as smart watches or fitness trackers. However, other wearables, such as smart garments, smart patches, glasses, jewelry, ear buds, etc, are also with the scope of this disclosure. Wearable device 102 can include one or more sensors for measuring the physiological variables and module for wirelessly communicating the data measured by the sensors (e.g., over Bluetooth) to an external device. (However, it is conceivable that wearable device 102 could store such data and transmit it when a wired connection to another device is made.)

As shown in FIG. 1, wearable device 102 can be connected to a mobile device 104, such as a smartphone or a tablet. It is, for example, typical for such devices to be paired with a smartphone for operation. Mobile device 104, in turn, can be connected, through an internet connection (e.g., through a Wi-Fi or a cellular connection) to one or more remote servers 106, where the early detection analysis can be performed. Alternatively, rather than a mobile device, wearable device 102 can be connected to any other suitable intermediate computing devices such as a laptop or desktop computer. In certain examples, wearable device 102 can connect directly to remote servers 106 (e.g. similarly through a Wi-Fi or a cellular connection), instead of through an intermediate device.

In certain alternative examples, rather than a remote server 106 (e.g., cloud computing) a connection to a local server can be used. For example, in a hospital setting, a local server, as part of a local patient management system, can be used to communicate with wearables worn by health workers and/or patients for early detection of an infection. In other examples, the remote or local server can be omitted, and the calculations can be performed on the wearable itself, or on the mobile or other computing device. Further, the early detection analysis can be distributed between multiple devices, such as a between wearable device 102 and remote server 106 or between mobile device 104 and remote server 106; other combinations are also conceivable.

FIG. 2 depicts a method 200 for early detection of an infection, using data collected by a wearable device. As described in connection with FIG. 1, the steps of the method 200 can be performed by a remote server receiving physiological data collected by a wearable device and transmitted from a device paired with the wearable or directly from the wearable; however, in various examples, the paired device, the wearable, or another computing device, such as a laptop or desktop computer or a local server can perform the analysis. Further, the steps of method 200 need not be performed by one device but can rather be distributed between multiple devices, including the wearable, the mobile device or other computing device, and the local or remote server. To that end, the steps of method 200 can be stored in one or more non-transitory storage media (e.g., located within the remote server or with another device performing the method) and executed by one or more processors in communication with the non-transitory storage medium.

At step 202, a time series (e.g., a vector) of data representing a physiological variable measured by a wearable device is received. The physiological variable can be any such variable useful for detecting the presence of an infection according to the following steps, such as, for example, body temperature, heart rate, and oxygen saturation. In practice, however, body temperature was found to be the most useful for early detection of infection. While this step presumes that some device other than the wearable device is performing the calculation, to the extent that one or more steps are performed on the wearable device, this step can be performed later in the method, or can be omitted altogether.

At step 204, a parametric function, defined by a set of parameters that include a skew and that characterize a distribution of a baseline window of the time series of data, can be determined. The baseline window can be a portion of the physiological data collected over a period of time sufficient to form a set of data representing values that are normal for a user (i.e., when the user is not carrying an infection). In an example, the baseline set of data can be collected over a ten-day period of time. However, confounding factors, such as short infections (e.g., the user contracts a small cold during the baseline window) can interfere with collecting the baseline data. To account for this, a much larger window, such as three months, can be used. In practice, a baseline window of twenty-one days was found to be sufficient to accurately determine a user's baseline.

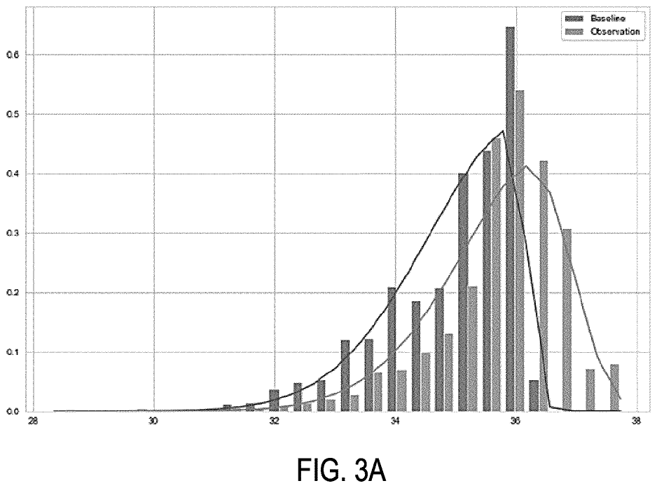

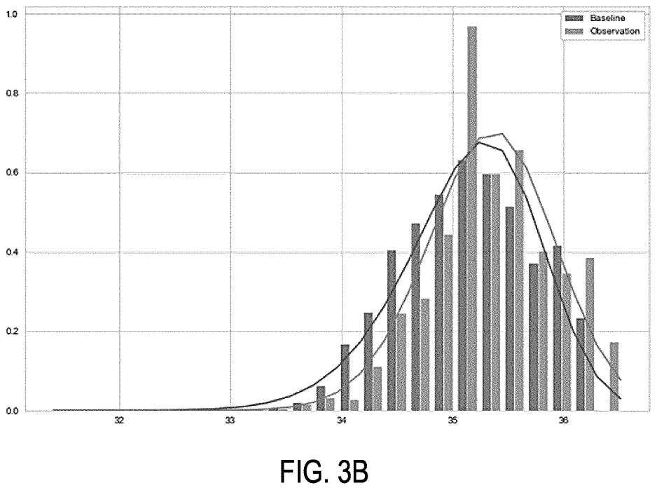

The baseline data set can be organized into a distribution, and a parametric function that accounts for the skew of the distribution can be fit to it. The distribution can be determined by assigning a value to each interval of the physiological value, representative of the total instances the interval occurs within the time series. Thus, taking temperature as an example, for each measured interval of temperature—e.g., 34°, 34.2°, 34.4°, 34.6° C.—a number, representing the number of times that temperature was measured, can be assigned to the interval. So, if, over the course of three months, a temperature of 34.8 is measured 1,500 times, the number 1500 would be assigned to 34.8. This data can then be normalized by the total number of measurements, resulting in a frequency per measurement that each interval occurs. Plotting this data would result in a histogram, such as shown in FIGS. 3A and 3B, discussed below.

A parametric function that characterizes the distribution of the measured temperature values can then be determined. As will be described in more detail below, the skew of the maximum likelihood fit curve (i.e., its measure of asymmetry of the distribution) is particularly useful for determining the presence of an infection, and so the parametric function selected can include a parameter than characterizes the skew of the function.

One such parametric function is the Skew Normal function, which can be defined, in an example, by Equation (1) below:

$$f ( x \vert μ , τ , α ) = 2 Φ ( ( x - μ ) \sqrt{τ} α ) ϕ ( x , μ , τ ) \qquad ( 1 )$$

where Φ is the cumulative density function, φ is the Gaussian density function, and μ and τ are the mean and inverse, respectively, of the standard deviation of the normal distribution. The additional parameter, α, is the skewness parameter; in the limit α=0, the gaussian distribution is recovered. Other equations for the Skew Normal function are known and are contemplated as alternatives. Further, other parametric functions that account for skewness of the distribution can be used, such as the Exponentially Modified Normal distribution.

At step 206, the method of step 204 can be repeated for an observation window of data, thus, a parametric function, defined by a set of parameters that include a skew and that characterize a distribution of an observation window of the time series of data, can be determined.

The observation window can be a portion of the physiological data collected over a period of time sufficient to detect that the user is carrying an infection when compared to the baseline window, as described in steps 208 and 210. In practice, a length of three days was found to be effective, however shorter (e.g., one days) or longer (e.g., four days) are contemplated. The observation window, however, typically cannot be longer than the infectious period, since data that is not relevant to an infected state can corrupt the data in the observation window.

It is generally preferable for the baseline window to terminate some period of time (e.g., three days) before the observation window begins, to avoid including data in the baseline window that could be indicative of an infectious state. However, it is contemplated that there can be some amount of overlap between the data forming the baseline window and the data forming the observation window, particularly where the baseline window is taken over a longer period of time, such as over several months.

Like step 204, the distribution can be normalized by the number of measurements taken in the observation window. The normalization of both the baseline measurement data and the observation measurement data permits comparison of the two windows, since the baseline window is typically much longer than the observation window, the total number of measurements within the baseline will be much higher, preventing meaningful comparison without normalization. Further, as in step 204, a parametric function that characterizes the distribution and accounts for skew, such as Skew Normal (as defined by Equation (1) or by other definitions in accepted in the art), or the Exponentially Modified Normal distribution, can be used to characterize the distribution.

An example of the distributions and the maximum likelihood fit, as defined by the Skew Normal function, of a time-series of a physiological variable—as described in connection with steps 204 and 206—are shown in FIGS. 3A and 3B. FIG. 3A, particularly shows the temperature distribution of temperatures for a COVID-19 positive user example. Specifically, the bars represent the distribution of the normalized baseline and observation window temperature values. Fit to these distributions are lines that represent Skew Normal functions obtained from a maximum likelihood fit to the data. FIG. 3B shows the Skew Normal functions characterizing the distributions of COVID-19 negative user baseline and observation windows. As shown, in FIG. 3B, the COVID-19 negative example, there is near total overlap between the Skew Normal functions. But in FIG. 3A, the COVID-19 positive example, Skew Normal function characterizing the observation window distribution is skewed to the right with respect to the Skew Normal function characterizing the baseline window distribution. There are also differences that can be noted in the peak values, with the baseline window having a higher peak than the observation window. It is these differences, and particularly the difference in skew, that can be leveraged by characterizing the distribution with a parametric function that includes a skewness value.

At step 208, whether the user is currently carrying an infection is determined from at least one measure of difference between the first set of parameters and the second set of parameters. Any suitable measure of difference of the parameters can be used, such as Kullback-Liebeler diverge, effect size, and earth mover's distance (also known as the Wasserstein metric), although other suitable metrics can be used. Effect size, for example, can be defined by Equation (2):

$$es = \frac{{μ}_{o b s} - {μ}_{b s l}}{\sqrt{( {σ}_{b s l}^{2} + {σ}_{o b s}^{2} ) / 2}} \qquad ( 2 )$$

where μ and σ are the means and standard deviations of the baseline and observation window functions, respectively. Notably, effect size only quantifies the difference between the gaussian components of the Skew Normal functions, whereas it is blind to differences in the corresponding tails. By contrast, Kullback-Liebeler diverge and earth mover's distance account for differences in the skew of the Skew Normal functions.

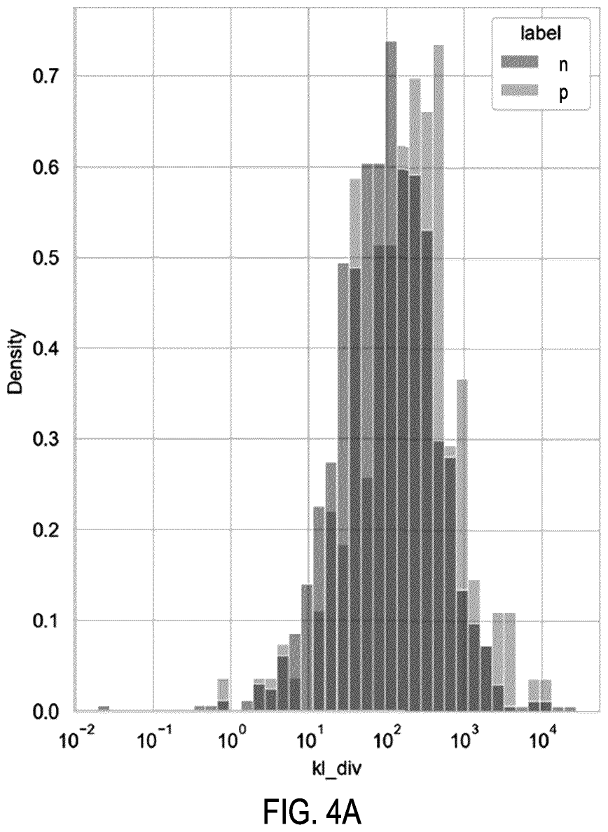

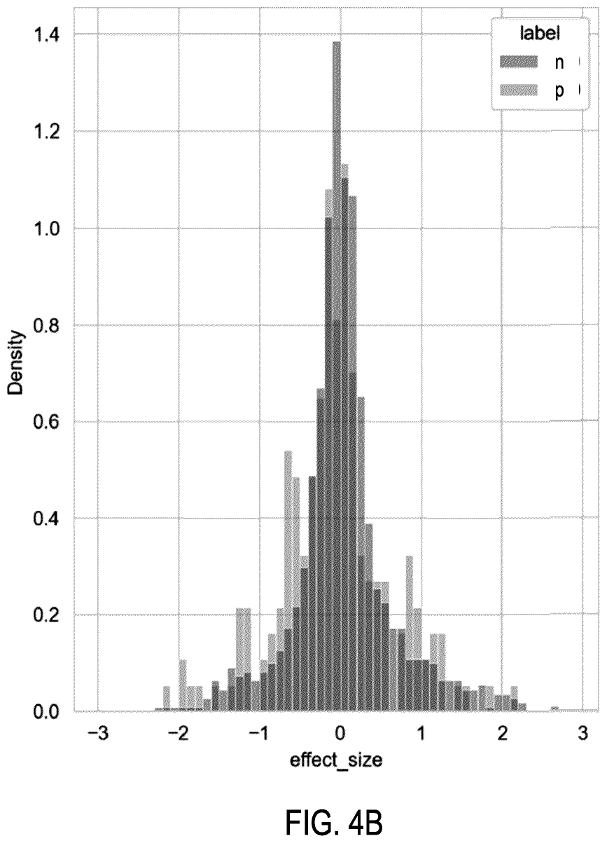

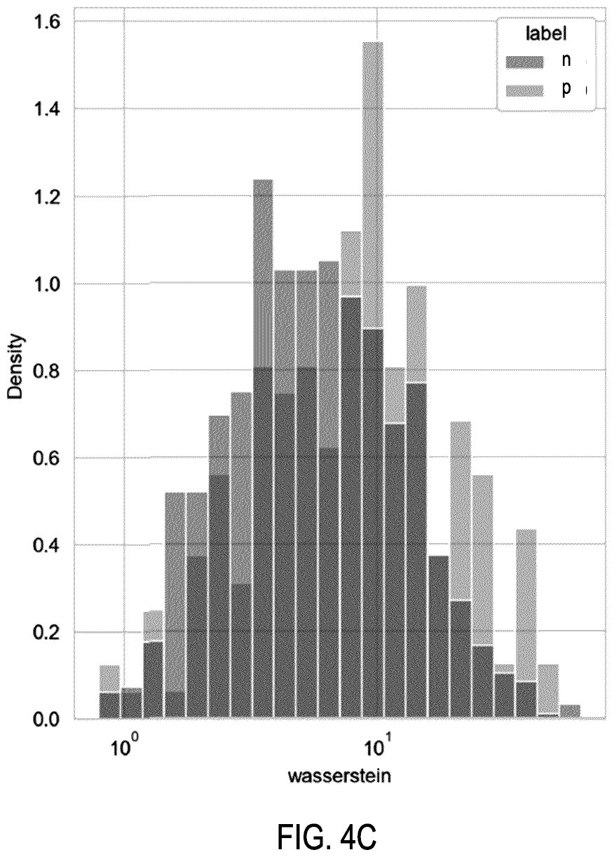

FIGS. 4A, 4B, and 4C depict distributions of the calculated values of Kullback-Liebeler diverge, effect size, and earth mover's distance, respectively, for a large number of COVID-19 negative users (denoted by n) and a large number of COVID-19 positive users (denoted by p). These distributions demonstrate consistent differences in values between COVID-19 negative and positive users for each measure of difference.

The measure(s) of difference in the parameters can be used to determine whether a given user is carrying an infection, such as COVID-19, by, for example, inputting the measure(s) of difference into a predictive model, such as a classification model (although other suitable predictive models could be used.) The predictive model will determine, from the one or more measures of difference, whether the given user is carrying an infection. Such predictive models are well known, and a suitable predictive model can be used.

At step 210, the user is alerted that the user is carrying an infection. The user can be alerted, for example, through a notification sent through the wearable itself (e.g., displayed on its screen or through or in combination with some haptic feedback) or through a mobile device or computer (e.g., a notification from a dedicated application, or a text message or email).

FIG. 5 depicts the steps of an example method 500 of determining the parametric function 204 (or 206) from a large set of data. More particularly, in the calculation of the parametric function of the baseline data, a large number of data points need to be stored in memory (often hundreds of megabytes) to form the distribution to which the parametric function is fit. This is memory and processing intensive. Accordingly, instead of storing all the data, the data can be chunked (i.e., separated into smaller portions) as it is received. Parametric functions of the smaller portions of data can then be determined as the data is received, with the parameters of the functions being stored rather than the data representative of the physiological variable. Once the parametric functions of the chunked baseline window are determined, the median or mean of the stored parameters can be used to approximate the parametric function of the larger dataset.

Thus, at step 502, the portion of data that forms the baseline is divided into smaller portions, as it is measured (e.g., every three days, although other lengths of time could be used). At step 504, the same parametric function fitting process described in step 204 is applied to the smaller portion of data, resulting in parametric function defined by a set of parameters, at least one of which describe the skew of the function fit to the smaller portion of data. The parameters can then be stored in place of retaining the portion of data in memory. This is repeated for each portion of data, such that a plurality of parametric functions are respectively fit to a plurality of portions of data as the data is measured, the parameters being stored for each in place of the data to which functions are fit. At step 506, the mean or the median of the stored are parameters are found. For example, if the Skew Normal is found for each portion, the following parameters are stored for each portion: u, t, a. Thus, the mean or median u, the mean or median t, and the mean or median a can be found. The resulting parameters of the mean or median represents an approximation of the parameters of the full dataset of the baseline window.

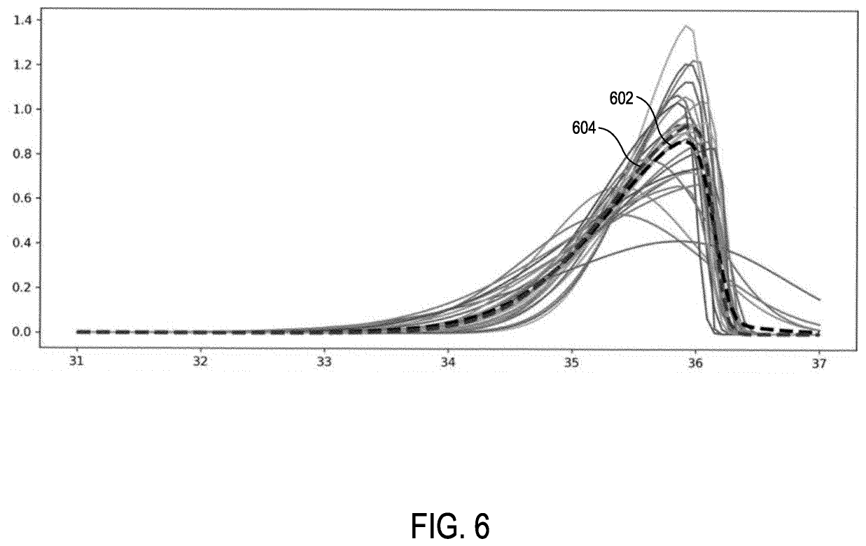

This can be seen in FIG. 6, in which shows an example set of maximum likelihood fits, respectively fit to temperature values measured over many three-day periods computed between one week and six months prior to a COVID-19 test for a single participant, as well as the mean and median values of the maximum likelihood fits. The dashed lines 602 and 604 represent maximum likelihood fits resulting from the mean and the median of these parameters, respectively.

The above-describe method and system can be deployed within a larger machine learning system that purposes user's physiological variables to provide advanced tracking of the user's health by leveraging a large dataset of thousands of similar users. For example, such a system—called RATE, for Rapid Analysis of Threat Exposure—has been developed by Philips North America (Cambridge, MA) in collaboration with the Department of Defense to monitor the well-being and readiness of soldiers, as reported in Scientific Reports journal by Nature Magazine on Mar. 8, 2022 (Conroy, B., Silva, I., Mehraei, G. et al. “Real-time infection prediction with wearable physiological monitoring and AI to aid military workforce readiness during COVID-19”. Sci Rep 12, 3797 (2022). The above-described method and system provides the additional benefit of allowing a personalized baseline to be developed for each user in addition to the large-scale analytics of the artificial intelligence system.

The present invention represents a considered improvement to the ability of a computer or a computer system to detect early infection of a user. Previous computers or computer systems failed due to the transient and marginal nature of the detected symptoms. The present invention thus provides a robust and repeatable method of early detection of infectious diseases, which is crucial to containing their spread or providing early treatment to the infected individual.

While this disclosure focuses on building features from temperature collected from wearable devices, the methodology described herein can be used on any other type of physiological measurement captured over an extended period of time. Moreover, while an ad-hoc parametric function has been used to model temperature, it is possible to perform a maximum likelihood fit over a wide range of parametric functions and then define a metric to automatically assign the best performing parametric function to a given the physiological input.

The present invention may be a system, a method, and/or a computer program product. The computer program product may include a non-transitory computer readable storage medium (or media) having computer readable program instructions thereon for causing a system or processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any combination of the foregoing, among other possibilities. Computer readable program instructions described herein can be downloaded to respective computing/processing devices from a computer readable storage medium or to an external computer or external storage device via a network, for example, the internet, a local area network, and/or a wireless network. Aspects of the present invention are described herein with reference to flowchart illustrations and/or block diagrams of methods, apparatus, systems, and computer program products according to embodiments of the invention. It will be understood that each block of the flowchart illustrations and/or block diagrams, and combinations of blocks in the flowchart illustrations and/or block diagrams, can be implemented by computer readable program instructions.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for determining, from data collected by a wearable device worn by a user, whether the user has an infection, comprising the steps of:
receiving, from the wearable device, a time series of data representing a physiological variable measured by the wearable device;
determining a first parametric function, defined by a first set of parameters, characterizing a distribution of a first portion of the time series of data, at least one of the first set of parameters characterizing a skew of the first parametric function;
determining a second parametric function, defined by a second set of parameters, characterizing a distribution of a second portion of the time series of data, at least one of the second set of parameters characterizing a skew of the second parametric function, wherein the first portion of the time series of data represents a baseline window and the second portion represents an observation window; and
determining from at least one measure of difference between the first set of parameters and the second set of parameters, whether the user is currently carrying an infection.

2. The method of claim 1, wherein determining a first parametric function, comprises:
dividing the first portion of the time series of data into a plurality of portions;
determining a plurality of parametric functions respectively characterizing a distribution of each of the plurality of the portions, wherein each of the plurality of parametric functions is defined by a set of parameters that characterize, at least, a skew of the parametric function, wherein the first parametric function is determined as a mean or a median of the set of parameters of the plurality of parametric functions.

3. The method of claim 1, wherein the first distribution function and the second distribution function are each skew normal functions.

4. The method of claim 1, wherein the at least one measure of difference between the first set of parameters and the second set of parameters, comprises determining at least one of a Kullback-Leibler divergence, effect size, and earth mover's distance.

5. The method of claim 1, wherein determining whether the user is currently bearing an infection comprises using the at least one measure of difference between the first set of parameters and the second set of parameters as an input to a predictive model.

6. The method of claim 5, wherein the predictive model is trained using data measured from a plurality of users.

7. The method of claim 5, wherein the predictive model is a classification model.

8. The method of claim 1, wherein the first portion of the time series of data is collected over at least three weeks.

9. The method of claim 1, wherein the second portion of the time series of data is collected over at most three days.

10. A non-transitory storage medium storing program code that, when executed by a processor, determines, from data collected by a wearable device worn by a user, whether the user has an infection, the program code comprising the steps of:
receiving, from the wearable device, a time series of data representing a physiological variable measured by the wearable device;
determining a first parametric function, defined by a first set of parameters, characterizing a distribution of a first portion of the time series of data, at least one of the first set of parameters characterizing a skew of the first parametric function;
determining a second parametric function, defined by a second set of parameters, characterizing a distribution of a second portion of the time series of data, at least one of the second set of parameters characterizing a skew of the second parametric function, wherein the first portion of the time series of data represents a baseline window and the second portion represents an observation window; and
determining from at least one measure of difference between the first set of parameters and the second set of parameters, whether the user is currently carrying an infection.

11. The non-transitory storage medium of claim 10, wherein determining a first parametric function, comprises:
dividing the first portion of the time series of data into a plurality of portions;
determining a plurality of parametric functions respectively characterizing a distribution of each of the plurality of the portions, wherein each of the plurality of parametric functions is defined by a set of parameters that characterize, at least, a skew of the parametric function, wherein the first parametric function is determined as a mean or a median of the set of parameters of the plurality of parametric functions.

12. The non-transitory storage medium of claim 10, wherein the first distribution function and the second distribution function are each skew normal functions.

13. The non-transitory storage medium of claim 10, wherein the at least one measure of difference between the first set of parameters and the second set of parameters, comprises determining at least one of a Kullback-Leibler divergence, effect size, and earth mover's distance.

14. The non-transitory storage medium of claim 10, wherein determining whether the user is currently bearing an infection comprises using the at least one measure of difference between the first set of parameters and the second set of parameters as an input to a predictive model.

15. The non-transitory storage medium of claim 14, wherein the predictive model is trained using data measured from a plurality of users.
